(19) United States
(12) Patent Application Publication
(10) Pub. No.: US 2019/0009068 A1
(43) Pub. Date: Jan. 10, 2019

(54) Methods And Applicators With Twist Tab And Resilient Structure For Medical Liquid Application

(71) Applicant: Razmik Margoosian, (US)

(72) Inventors: Razmik Margoosian, Montreal (CA); Viken Afarian, Laval (CA)

(73) Assignee: Razmik Margoosian, Montreal (CA)

(21) Appl. No.: 15/757,198

(22) PCT Filed: Sep. 2, 2016
(86) PCT No.: PCT/CA2016/051050

§ 371 Date: Jun. 1, 2018

Related U.S. Application Data
(60) Provisional application No. 62/213,349, filed on Sep. 2, 2015.

(51) Int. Cl. A61F 13/40; A61J 1/06

(52) U.S. Cl. CPC A61M 35/006; A61J 1/065

(57) ABSTRACT

An applicator can have an ampoule body for holding a medical liquid; a tab extending away from the ampoule body and being twistable or deflectable with respect to a longitudinal axis of the ampoule body; and a frangible region in between the tab and the distal portion of the ampoule body, the frangible region being breakable in response to twisting or deflection of the tab. The applicator can have a resilient structure extending between the tab and the ampoule body and configured to allow the twisting or deflection to enable breakage of the frangible region while remaining intact and providing axial rigidity in response to a deflection pressure exerted on the tab during application of the medical liquid. The applicator can have features such as a thumb rest, a bubble portions, a pyramidal body construction, an offset frangible nodule, and various tab and head constructions.

### TECHNICAL FIELD

The technical field generally relates to liquid application onto a surface, and more particularly to liquid dispensing applicators for applying medical liquids to the surface of patients, as well as methods of manufacturing such liquid dispensing applicators.

### BACKGROUND

Applicators for applying liquids to surfaces are broadly used in several industries such as the medical domain to treat or clean. Hygiene, reliability, ease of use and safety are some of the desirable characteristics for such applicators.

Some known applicators have an ampoule that holds the medical liquid and a twist off tab attached to a tip of the ampoule. When the tab is twisted off by a user, a frangible region in between the tab and the ampoule breaks to enable fluid flow. Once twisted, the tab is completely broken off of the ampoule or can remain fragilely attached via a very thin plastic membrane.

Known medical liquid applicators have a number of disadvantages related to reliability, ease of use, safety, manufacturing and effective medical liquid application. There is a need for a medical liquid applicator that overcomes at least some of the disadvantages of what is known in this field.

### SUMMARY

In some implementations, there is provided a medical liquid applicator comprising:
an ampoule body comprising a proximal portion and a distal portion, and having a wall defining a chamber for holding a medical liquid;
a tab attached to and extending away from the distal portion of the ampoule body, the tab being twistable about or deflectable with respect to a longitudinal axis of the ampoule body;
a frangible region in between the tab and the distal portion of the ampoule body, the frangible region being breakable in response to twisting or deflection of the tab to form an opening in fluid communication with the chamber to allow liquid communication out of the chamber into an absorbent material connectable to the tab; and
a resilient structure extending between the tab and the ampoule body and configured to allow the twisting or deflection of the tab to enable breakage of the frangible region while remaining intact and providing axial rigidity to resist axial deflection of the tab in response to a deflection pressure exerted on the tab during application of the medical liquid.

In some implementations, the resilient structure comprises at least two resilient structures provided on opposed lateral sides of the tab and connecting to opposed lateral sides of the distal portion of the ampoule.

In some implementations, the resilient structures each comprise an arm having a first portion extending from the tab along and in spaced-apart relation to the distal portion of the ampoule and a second portion extending toward and attaching to the distal portion of the ampoule.

In some implementations, the resilient structures each comprise a thick zone integrally formed with the tab and the distal portion of the ampoule body.

In some implementations, the thick zone has a thickness of about 1.5 to about 3 times the thickness of the wall.

In some implementations, the applicator is formed as an integral one-piece unit. In some implementations, the integral one-piece unit is made by a blow-fill-seal (BFS) technique.

In some implementations, the tab further comprises at least one spike oriented to aid in retaining the absorbent material thereon.

In some implementations, the ampoule body is generally tubular. In some implementations, the tab is generally flat.

In some implementations, the resilient structure is configured to be non-deformable upon twisting of the tab.

In some implementations, the resilient structure is configured to return to a substantially pre-twisting position after twisting the tab and breaking the frangible region.

In some implementations, the resilient structure is composed of HDPE or PP.

In some implementations, the medical liquid is heat sterilized.

In some implementations, the medical liquid comprises chlorhexidine.

In some implementations, the medical liquid comprises chlorhexidine gluconate.

In some implementations, the applicator further comprises the absorbent material that is affixed to with respect to the tab. The sponge may alternatively be provided separately or as part of a kit.

In some implementations, the absorbent material comprises a sponge have opposed sides and a cavity housing the tab.

In some implementations, part of the sponge is heat-bonded to part of the tab and/or part of the distal portion of the ampoule body.

In some implementations, the sponge is further retained on the tab by engaging with retention elements provided on the sponge.

In some implementations, the retention elements include spikes extending from a surface of the tab.

In some implementations, at least a portion of the spikes are provided along side edges of the tab.

In some implementations, all parts are composed of HDPE or PP, except for the absorbent material.

In some implementations, there is provided a method of making a medical liquid applicator with an affixed sponge, comprising:
using a blow-fill-seal technique to make the medical liquid applicator;
heat treating at least part of the medical liquid applicator to provide heat treated regions;
applying the sponge such that portions of the sponge contact and bond with the heat treated regions of the medical liquid applicator.

In some implementations, the step of heat treating is done by contacting the corresponding regions of the medical liquid applicator with a heating element.

In some implementations, the step of heat treating comprises heating opposed sides of the applicator, and the step of applying the sponge comprises providing two sponge strips that contact respective heat treated regions on opposed sides of the applicator and then cutting and heat-sealing the two sponge strips to form edges and define a sponge cavity in which the tab in located.

In some implementations, the cutting and heat-sealing are performed by the same equipment.

In some implementations, a plurality of the applicators are conveyed on a conveyor between each step.

In some implementations, the method further comprises heat sterilizing the medical liquid in the applicator.

In some implementations, the heat sterilizing is conducted prior to the heat treating step.

In some implementations, the applicator is as defined above or herein.

In some implementations, the BFS technique can be replaced by other molding techniques.

In some implementations, there is provided a medical liquid applicator, comprising:
an ampoule body comprising a proximal portion and a distal portion, and having a wall defining a chamber for holding a medical liquid;
a tab attached to and extending away from the distal portion of the ampoule body, the tab being twistable about or deflectable with respect to a longitudinal axis of the ampoule body;
a frangible region in between the tab and the distal portion of the ampoule body, the frangible region being breakable in response to twisting or deflection of the tab to form an opening in fluid communication with the chamber to allow liquid communication out of the chamber into an absorbent material connectable to the tab; and
a guide comprising:
   a guide body defining a cavity configured for receiving at least the tab upon insertion; and
   a guide mechanism located within the guide body and configure to contact the tab and cause the rotation or the deflection of the tab in response to axial insertion of the tab into the guide body;

In some implementations, the applicator further comprises a resilient structure extending between the tab and the ampoule body and configured to allow the twisting or deflection of the tab to enable breakage of the frangible region while remaining intact and providing axial rigidity to resist axial deflection of the tab in response to a deflection pressure exerted on the tab during application of the medical liquid.

In some implementations, the guide is configured such that, once the frangible region has broken, the guide and the ampoule body remain fixed together, and the guide body provides axial rigidity to resist axial deflection in response to a deflection pressure exerted on the guide during application of the medical liquid

In some implementations, the applicator further comprises a locking mechanism for locking the tab relative to the guide mechanism once the frangible region has broken. In some implementations, the locking mechanism comprises a projection and a corresponding recess. The locking mechanism and the guide body can have various constructions and features as described herein

In some implementations, the applicator can be made by the methods described above or herein. The applicator with guide can also have various additional features as described above or herein.

In some implementations, there is provided a medical liquid applicator, comprising:
an ampoule body comprising a proximal portion and a distal portion, and having a wall defining a chamber for holding a medical liquid;
a tab attached to and extending away from the distal portion of the ampoule body, the tab being twistable about or deflectable with respect to a longitudinal axis of the ampoule body;
a frangible region in between the tab and the distal portion of the ampoule body, the frangible region being breakable in response to twisting or deflection of the tab to form an opening in fluid communication with the chamber to allow liquid communication out of the chamber into an absorbent material connectable to the tab; and
a guide comprising:
   a guide body defining a cavity configured for receiving at least the tab upon insertion; and
   a guide mechanism located within the guide body and configure to contact the tab and cause the rotation or the deflection of the tab in response to axial insertion of the tab into the guide body.

In some implementations, the medical liquid applicator further includes a resilient structure extending between the tab and the ampoule body and configured to allow the twisting or deflection of the tab to enable breakage of the frangible region while remaining intact and providing axial rigidity to resist axial deflection of the tab in response to a deflection pressure exerted on the tab during application of the medical liquid.

In some implementations, the guide is configured such that, once the frangible region has broken, the guide and the ampoule body remain fixed together, and the guide body provides axial rigidity to resist axial deflection in response to a deflection pressure exerted on the guide during application of the medical liquid

In some implementations, the applicator includes a locking mechanism for locking the tab relative to the guide mechanism once the frangible region has broken. In some implementations, the locking mechanism comprises a projection and a corresponding recess.

In some implementations, there is provided a medical liquid applicator, comprising: an ampoule body comprising a proximal portion and a distal portion, and having a wall defining a chamber for holding a medical liquid; a tab attached to and extending away from the distal portion of the ampoule body, the tab being twistable about or deflectable with respect to a longitudinal axis of the ampoule body; a frangible region in between the tab and the distal portion of the ampoule body, the frangible region being breakable in response to twisting or deflection of the tab to form an opening in fluid communication with the chamber to allow liquid communication out of the chamber; and a thumb rest disposed on an upper side of the ampoule body and comprising an upward-facing surface having a concave curvature.

In some implementations, the thumb rest comprises ridges disposed on the upward-facing surface. In some implementations, the ridges are arranged to extend laterally across a forward region of the thumb rest. In some implementations, a rearward-most ridge is provided at a lowest part of the curvature, and the forward-most ridge is provided at a highest part of the curvature. In some implementations, the thumb rest comprises a rearward end and a forward end, the forward end comprising a lip that is raised with respect to adjacent external surfaces of the ampoule body. In some implementations, the rearward end is configured to join in flush fashion with adjacent external surfaces of the ampoule body. In some implementations, the curvature is continuous and constant. In some implementations, the curvature has an arc of 10° to 40°, or 20° to 30°. In some implementations, the thumb rest has a generally oval or stadium shape viewed from above. In some implementations, the ampoule body has a flat upper wall and the thumb rest is disposed on the flat upper wall.

In some implementations, there is provided a medical liquid applicator, comprising: an ampoule body comprising a proximal portion and a distal portion, and having a wall defining a chamber for holding a medical liquid; a tab attached to and extending away from the distal portion of the ampoule body, the tab being twistable about or deflectable with respect to a longitudinal axis of the ampoule body; a frangible region in between the tab and the distal portion of the ampoule body, the frangible region being breakable in response to twisting or deflection of the tab to form an opening in fluid communication with the chamber to allow liquid communication out of the chamber; and at least one bubble portion provided on an bottom side of the ampoule body, each bubble portion comprising a bubble wall that is continuous with the wall of the ampoule body and projects to form an elongated bubble compartment that is configured to facilitate fluid dispending in response to compression of the ampoule body.

In some implementations, the ampoule body comprise a main section having a generally triangular cross-section so as to provide an upper wall and two bottom inclined walls, and the at least one bubble portion comprise two bubble portions arranged on the two bottom inclined walls. In some implementations, each bubble portion is generally stadium shaped. In some implementations, each bubble portion is provided in opposed relation to a thumb rest. In some implementations, each bubble portion extends the entire length of the thumb rest. In some implementations, each bubble portion has a volume between 50 mm³ and 300 mm³ or between 100 mm³ and 200 mm³.

In some implementations, there is provided a medical liquid applicator, comprising:
a body region comprising a main container portion having a wall defining a chamber for holding a medical liquid;
a head region coupled to the body region and comprising:
   a neck portion extending forwardly from a distal end of the main container portion and defining a neck chamber in fluid communication with the chamber of the main container portion;
   a cellular tab unit coupled to and extending forwardly away from a distal end of the neck portion;
   a frangible nodule located in between the cellular tab unit and the neck portion, and being breakable in response to rotation of the cellular tab unit relative to the neck portion to form an opening in the neck portion to allow the medical liquid to flow there-through;
   a first lateral cellular unit and a second lateral cellular unit disposed on opposing sides of the neck portion and extending lengthwise along at least a part of the neck portion;
   a first rigid web disposed between and joining the first lateral cellular unit to the neck portion and the main container portion;
   a second rigid web disposed between and joining the second lateral cellular unit to the neck portion and the main container portion; and
   a frangible web extending from a lateral edge of the head region above the first lateral cellular unit, inwardly to the frangible nodule, the frangible web being tearable in response to rotation of the cellular tab unit relative to the neck portion such that upon rotation:
      the frangible web tears from the lateral edge to the frangible nodule; and then
      the frangible nodule breaks to form the opening in the neck portion for fluid flow out therefrom.

In some implementations, the first lateral cellular unit comprises a distal extremity that is located rearward of the neck portion. In some implementations, the head region further comprises a third lateral cellular unit located on a distal side of the frangible web; and a third rigid web coupling the third lateral cellular unit to the cellular tab unit. In some implementations, the second lateral cellular unit extends longitudinally from the main container portion to the cellular tab unit and is connected to the cellular tab unit by a forth rigid web. In some implementations, the head region further comprises a secondary frangible web extending from the frangible nodule in between a distal corner of the neck portion and a distal inner side of the second lateral cellular unit. In some implementations, the frangible web defines a zigzag path from the lateral edge to the frangible nodule, and/or may have features as described or illustrated herein. In some implementations, the first and second lateral cellular units have generally straight outer walls and inner walls that are shaped to follow an outer contour of the neck portion. In some implementations, the first and second lateral cellular units each have proximal portions and distal portions, the proximal portions being wider than the distal portions. In some implementations, the cellular tab unit comprises a distal part and a proximal part, the distal part being wider than the proximal part. In some implementations, the distal part has a generally circular segment shape viewed from above with a forward curved surface, and the proximal part has a generally circular segment shape viewed from above with a rearward curved surface. In some implementations, each of the cellular tab unit, the first lateral cellular unit, and the second lateral cellular unit is substantially symmetrical bout a longitudinal cross-sectional plane extending through the applicator. In some implementations, the head region and the body region together have generally continuous lateral edges viewed from above. In some implementations, each of the cellular tab unit, the first lateral cellular unit, and the second lateral cellular unit has a single-cell structure.

In some implementations, there is provided a medical liquid applicator, comprising: an ampoule body comprising a proximal portion and a distal portion, and having a wall defining a chamber for holding a medical liquid, wherein the ampoule body comprises a main body section having a triangular cross-section; a tab attached to and extending away from the distal portion of the ampoule body, the tab being twistable about or deflectable with respect to a longitudinal axis of the ampoule body; and a frangible region in between the tab and the distal portion of the ampoule body, the frangible region being breakable in response to twisting or deflection of the tab to form an opening in fluid communication with the chamber to allow liquid communication out of the chamber.

In some implementations, the main body section comprises an upper wall and two bottom inclined walls. In some implementations, the upper wall and the two bottom inclined walls have substantially equal widths to each other. In some implementations, the ampoule body comprises a rear region and a forward region each coupled to respective ends of the main body section, and wherein the forward region has an inclined bottom wall that tapers upwardly in a forward direction. In some implementations, the rear region has an inclined bottom wall that tapers upwardly in a rearward direction.

In some implementations, there is provided a medical liquid applicator, comprising: an ampoule body comprising a proximal portion and a distal portion comprising an ampoule neck having bulbous portion with a forward-facing surface, and having a wall defining a chamber for holding a medical liquid; a tab attached to the ampoule neck, the tab being twistable about or deflectable with respect to a longitudinal axis of the ampoule body, the tab comprising a bulbous part with a rearward-facing surface; and a frangible region in between the tab and the ampoule neck, the frangible region being breakable in response to twisting of the tab to form an opening in fluid communication with the chamber to allow liquid communication out of the chamber; wherein the forward-facing surface of the bulbous portion of the ampoule neck and the rearward-facing surface of the bulbous part of the tab are positioned and configured to abut on each other in response to axial deflection of the tab after breakage of the frangible region.

In some implementations, the bulbous portion and the bulbous part are configured and positioned to contact upon deflection of 5° and 40° of the tab relative to the ampoule body, and to prevent further deflection. In some implementations, the bulbous portion and the bulbous part are configured and positioned to contact upon deflection of 5° and 30° of the tab relative to the ampoule body, and to prevent further deflection. In some implementations, the bulbous portion and the bulbous part are configured and positioned to contact upon deflection of 10° and 20° of the tab relative to the ampoule body, and to prevent further deflection. the bulbous portion and the bulbous part are hollow. In some implementations, the bulbous portion and the bulbous part are each symmetrical about a longitudinal cross-section. In some implementations, the bulbous portion and the bulbous part have a substantially similar height.

In some implementations, there is provided a medical liquid applicator, comprising: an ampoule body comprising a proximal portion and a distal portion, and having a wall defining a chamber for holding a medical liquid; a tab attached to and extending away from the distal portion of the ampoule body, the tab being twistable about or deflectable with respect to a longitudinal axis of the ampoule body; and a frangible region in between the tab and the distal portion of the ampoule body, the frangible region being breakable in response to twisting or deflection of the tab to form an opening in fluid communication with the chamber to allow liquid communication out of the chamber, wherein the frangible region has a center that is positioned offset with respect to a central longitudinal axis of the ampoule body and the tab.

In some implementations, the frangible region comprises a frangible nodule. In some implementations, the frangible nodule has a generally circular cross-section. In some implementations, none of the frangible region crosses the central longitudinal axis. In some implementations, a portion of the frangible region crosses the central longitudinal axis.

In some implementations, there is provided a medical liquid applicator, comprising:
a body region comprising a main container portion having a wall defining a chamber for holding a medical liquid;
a head region coupled to the body region and comprising:
   a neck portion extending forwardly from a distal end of the main container portion and defining a neck chamber in fluid communication with the chamber of the main container portion;
   a tab coupled to and extending forwardly away from a distal end of the neck portion;
   a frangible nodule located in between the tab and the neck portion, and being breakable in response to rotation of the tab relative to the neck portion to form an opening in the neck portion to allow the medical liquid to flow there-through;
   a first lateral unit and a second lateral unit disposed on opposing sides of the neck portion and extending lengthwise along at least a part of the neck portion, the second lateral unit forming a resilient structure extending between the tab and the ampoule body and configured to allow the twisting of the tab to enable breakage of the frangible region while remaining intact;
   a frangible web extending from a lateral edge of the head region above the first lateral unit, inwardly to the frangible nodule, the frangible web being tearable in response to rotation of the cellular tab unit relative to the neck portion such that upon rotation:
      the frangible web tears from the lateral edge to the frangible nodule; and then
      the frangible nodule breaks to form the opening in the neck portion for fluid flow out therefrom.

In some implementations, the first lateral unit comprises a distal extremity that is located rearward of the neck portion. In some implementations, the head region further comprises a third lateral unit located on a distal side of the frangible web, and coupled to the tab. In some implementations, the second lateral unit extends longitudinally from the main container portion to the tab and is rigidly connected to the tab. In some implementations, the head region further comprises a secondary frangible web extending from the frangible nodule in between a distal corner of the neck portion and a distal inner side of the second lateral unit.

In some implementations, the frangible web defines a zigzag path from the lateral edge to the frangible nodule. In some implementations, the frangible web comprises: a first frangible portion extending laterally from the lateral edge to a side part of the neck portion, a second frangible portion extending along the side part of the neck portion, and a third frangible portion extending along a distal part of the neck and terminating at the frangible nodule. In some implementations, the first and third frangible portions are parallel to each other and perpendicular to the second frangible portion. In some implementations, the first, second and third frangible portions have lengths that are within 30% of each other. In some implementations, the first, second and third frangible portions have lengths that are within 20% of each other. In some implementations, the first, second and third frangible portions have lengths that are within 10% of each other. In some implementations, the first and second frangible portions join at a curved bend. In some implementations, the second and third frangible portions join at a curved bend. In some implementations, the first, second and third frangible portions are each generally straight. In some implementations, the longitudinal position of the first frangible portion can be provided to facilitate ease of tearing in response to rotation of the tab.

In some implementations, the first and second lateral units are shaped to follow an outer contour of the neck portion. In some implementations, the first and second lateral units each have proximal portions and distal portions, the proximal portions being wider than the distal portions. In some implementations, the first and second lateral units each have a cellular structure.

In some implementations, the applicator further includes a head component that includes a slot sized and configured to receive at least a part of the tab such that rotation of the head component relative to the tab causes rotation of the tab and breakage of the frangible region, the head component including a channel allowing fluid flow from the opening to a sponge disposed on the head component. In some implementations, the applicator further includes a radial locking mechanism and/or an axial locking mechanism for locking the head component to the ampoule body.

In some implementations, the applicator can be manufactured by at least one of blow molding, blow-fill-seal (BFS) molding, or injection molding, or a combination thereof. In some implementations, the applicator is composed of HDPE, PET, PP or a combination thereof.

In some implementations, the medical liquid dispensing applicator of the previous paragraph comprises one or more features as described in any one or a combination of the above paragraphs, the drawings and/or the below description, such as a sound-generating system, one or more wing elements, a ratchet system, one or more locking systems, and/or a certain configuration of one or more projections and corresponding slot system.

### BRIEF DESCRIPTION OF THE DRAWINGS

FIG. 1 is a front cut view schematic of part of a medical liquid applicator.

FIG. 2 is another front cut view schematic of part of a medical liquid applicator.

FIG. 3 is another front cut view schematic of part of a medical liquid applicator.

FIG. 4 is another front cut view schematic of part of a medical liquid applicator.

FIG. 5 is a partial front cut, partial transparent view schematic of part of a medical liquid applicator and an absorbent material.

FIG. 6 is a front view schematic of part of a medical liquid applicator.

FIG. 7 is a side view schematic of part of a medical liquid applicator.

FIG. 8 is a process diagram.

FIG. 9 is a front view schematic of part of a tab.

FIG. 10 is a side view schematic of part of a tab.

FIG. 11a is a side view schematic of part of a medical iquid applicator and a heating element for heat treatment. The heating can be done by contact or by remote heating methods.

FIG. 11b is a side view schematic of part of the medical liquid applicator including heat treated regions and sponge strips.

FIG. 11c is a side view schematic of part of the medical liquid applicator and sponge strips applied to the heated treated regions.

FIG. 11d is a side view schematic of part of the medical liquid applicator and a sponge that has been cut and heat-sealed around the tab.

FIG. 12 is a graph of stress versus strain.

FIGS. 13a and 13b are side view schematics illustrating bending of the resilient structures when in use.

FIG. 14 is a side cut view schematic of a tab, a guide and a sponge.

FIGS. 15a to 15g are side cut view schematics of various guide configurations.

FIG. 16 is a side view of part of a tab and a guide mechanism.

FIG. 17 is a top plan view of an applicator.

FIG. 18 is a side plan view of an applicator.

FIG. 19 is a front plan view of an applicator.

FIG. 20 is a bottom perspective view of an applicator.

FIG. 21 is a top plan close-up view of an applicator head region.

FIG. 22 is a top plan close-up schematic view of an applicator head region.

FIG. 23 is a cross-sectional view of part of an applicator head region showing cellular units.

FIG. 24 is a top plan view of an applicator with a head component.

### DETAILED DESCRIPTION

The medical liquid applicator can include an ampoule body for holding medical liquid, a twistable tab, a frangible region in between the tab and the ampoule body which breaks in response to twisting the tab, and a resilient structure that extends between the tab and the ampoule body and is configured to allow the twisting of the tab while remaining intact and also providing axial stiffness or rigidity to resist axial deflection of the tab in response to a deflection pressure exerted on the tab during application of the medical liquid onto a surface. The resilient structure thus provides the structural resistance to axial deflection to enable application of the medical fluid immediately after the frangible region is broken to enable fluid to be dispensed. The resilient structure can provide the necessary rigidity between the ampoule body that is held in the hand of a user and the tab over which a sponge can be provided to facilitate applying and spreading the medical liquid.

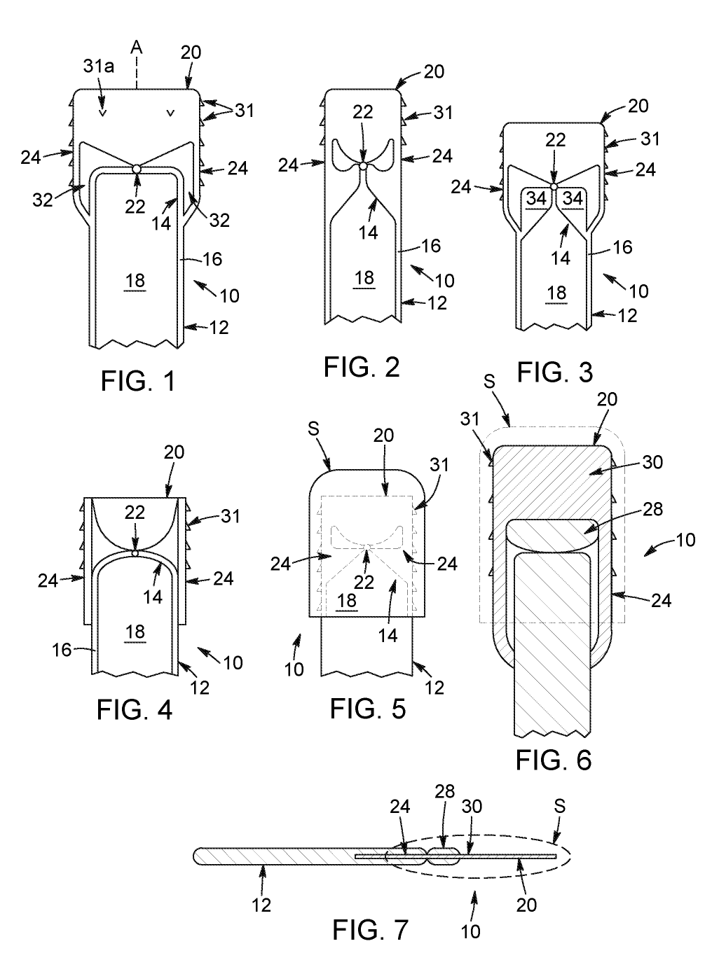

Referring to FIGS. 1 to 4, the medical liquid applicator 10 includes an ampoule body 12 that has a proximal portion (not shown) and a distal portion 14 and having a wall 16 that defines a chamber 18 for holding the medical fluid (not shown). The wall 18 can be composed of a polymer, such as high density polyethylene (HDPE) or polypropylene (PP), and may have a thickness between 0.5 mm and 1 mm, optionally between 0.6 mm and 0.9 mm or between 0.7 mm and 0.8 mm, for example. Preferably, the material and thickness are provided to prevent any kinking of the ampoule body during hand-held application. The ampoule body 12 may have various forms and structures and may be configured to enable a user to hold the body by hand and enable squeezing to force the fluid out of the chamber when possible. Preferably, the ampoule body is generally tubular and may have a central portion that is substantially cylindrical.

The medical liquid applicator 10 also includes a tab 20 that extends outwardly from the distal portion 14 of the ampoule body 12. The tab 20 is sized and configured to be twisted or otherwise deflected or displaced by a user, and can thus include parts that have a generally flat configuration. The tab can includes different regions having different thicknesses and functionalities, as will be described further below.

The medical liquid applicator 10 also includes a frangible region 22 located in between the tab 20 and a central extremity of the distal portion 14 of the ampoule body 12. The frangible region 22 is configured such that when the tab 20 is twisted and/or deflected the frangible region 22 breaks and forms an opening in the chamber to allow liquid communication out of the chamber 18. The frangible region 22 may be sized and configured to form the opening having a diameter of 0.075 mm to 0.15 mm, optionally between 0.09 mm and 0.12 mm.

Referring to FIGS. 1 to 7, the medical liquid applicator 10 also includes a resilient structure 24 extending between the tab 20 and the ampoule body 12. The resilient structure 24 is sized, positioned and configured to allow the rotation of the tab 20 for breakage of the frangible region while remaining intact and providing axial structural properties to resist axial deflection of the tab 20. Thus, when the tab 20 is twisted or deflected to cause breakage of the frangible region, the resilient structure keeps the tab and the ampoule body connected and then provides axial strength to resist deflection pressures exerted on the tab 20 during application of the medical liquid.

Referring briefly to FIGS. 5 to 7, an absorbent material 26, such as a sponge, can be provided surrounding the tab 20 and possibly part of the distal portion of the ampoule body 12. The sponge 26 can receive the liquid released from the chamber and facilitate application of the fluid to a patient or other surface. In operation, a user holds the ampoule body 12 in their hand and presses the sponge against the surface which causes an axial deflection force (i.e., with respect to the longitudinal axis of the ampoule body) to be exerted against the tab 20. The resilient structure 24 provides the structural properties to resist such axial deflection forces in order to facilitate application of the liquid. Without the resilient structure, the tab 20 would not be able to provide sufficient structure to support the sponge 26.

Referring to FIGS. 1 to 7, the resilient structure 24 can have various constructions and configurations. Optionally, the resilient structure 24 includes at least two resilient structures on opposed sides of the tab 20 which can facilitate providing symmetrical reinforcement and structural properties. In some implementations, the resilient structure 24 is configured to account for a certain degree of twisting or rotation of the tab to achieve breakage of the frangible region, for example by having sufficient structural resilience to avoid deforming upon twisting the tab 90 degrees or having sufficient structural resilience such that after twisting the tab 90 degrees the resilient structure maintains sufficient resistance to axial deflection.

FIGS. 1, 3, 4 and 6 illustrates a scenario where the resilient structure 24 includes side arms that extend from opposed sides of the tab 20 in spaced-apart relation to part of the wall of the ampoule body, and then attach to the wall 16 of the distal portion 14 of the ampoule body 12. FIGS. 2 and 5 illustrate a scenario where the resilient structure 24 includes structural regions that extend from opposed lower side ends of the tab 20 and join the upper side ends of the distal portion 14 of the ampoule body 12. It should be noted that the resilient structure 24—whether in the form of side arms, structural regions, or another form—may include regions of thicker or denser polymer material, different material from the rest of the tab and ampoule, and/or a shape or configuration that enables the structural features of enabling tab twisting while providing axial strength or rigidity and resistance to axial deflection.

Referring now to FIGS. 6 and 7, the tab 20 may include different regions that have different configurations. For instance, the tab 20 may include a thick region 28 and a thin region 30. In some implementations, the thin region 30 is the same thickness and is generally continuous with the resilient structures 24, which can be about 1.5 to 3 times the thickness of the wall of the ampoule body, optionally about 2 times the thickness.

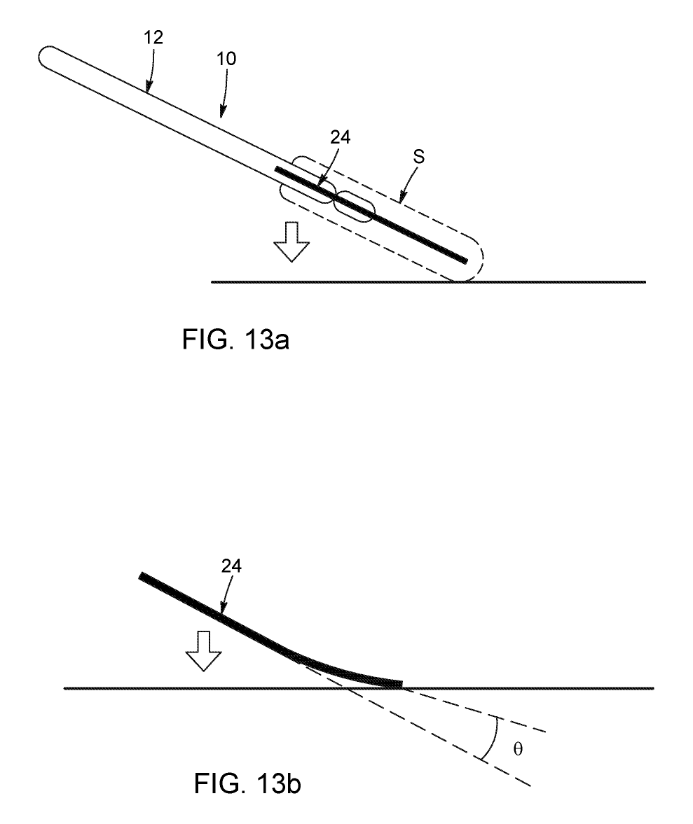

In some implementations, the resilient structures 24 are sized, shaped, configured and composed of a material in order to achieve desired elasticity and rigidity properties. In some scenarios, the resilient structures 24 can undergo elastic deformation under the torsion force when the tab is twisted, and does not undergo any plastic deformation or hardening, thereby returning to the original shape and position after the tab is twisted. Referring to FIG. 12, the resilient structures 24 (and preferably the other components of the applicator except for the frangible region) remain in the elastic deformation zone of the stress-strain curve. In some scenarios, after twisting the resilient structures 24 experience no loss of axial rigidity. In some scenarios, the resilient structures 24 can undergo elastic deformation under the bending force when a user is applying the medical liquid to a surface. Optionally, the resilient structures each have properties (e.g., density, structure, shape, etc.) such that the angle θ, as illustrated in FIG. 13b due to bending strain experienced during application of the medical fluid, is 0° to 45°, 2° to 30°, 5° to 20° or 10° to 15°, for example.

In some implementations, when the tab is twisted, the resilient structures remain substantially non-deformed due to high rigidity while adjacent regions of the applicator experience some degree of strain and deformation. Preferably, the adjacent regions are configured to experience elastic deformation only in response to the torsional force applied during tab twisting. In this regard, it should be noted that the resilient structures and the adjacent regions may have different combinations of properties in order to enable the tab twisting and the axial rigidity. For example, if the resilient structures have very high axial rigidity, the adjacent regions would tend to experience higher strain, in which case the adjacent regions would preferably be provided with sufficient elasticity to avoid or limit permanent deformation. If the resilient structures have lower axial rigidity, then resilient structures can experience more deformation during the twisting and the adjacent regions experience less as deformation is more distributed.

Referring to FIGS. 1 to 6, the tab 20 may include at least one spike 31 or pique that can contact and help retain the sponge. In some implementations, each side of the tab is provided with multiple spaced-apart edge spikes 31. It is understood that the number, length and orientation of edge spikes 31 per side may vary. The edge spikes 31 can facilitate to holding the absorbent material in place, such that no or only a small amount of adhesive material is required to prevent the absorbent material from being easily removed during application.

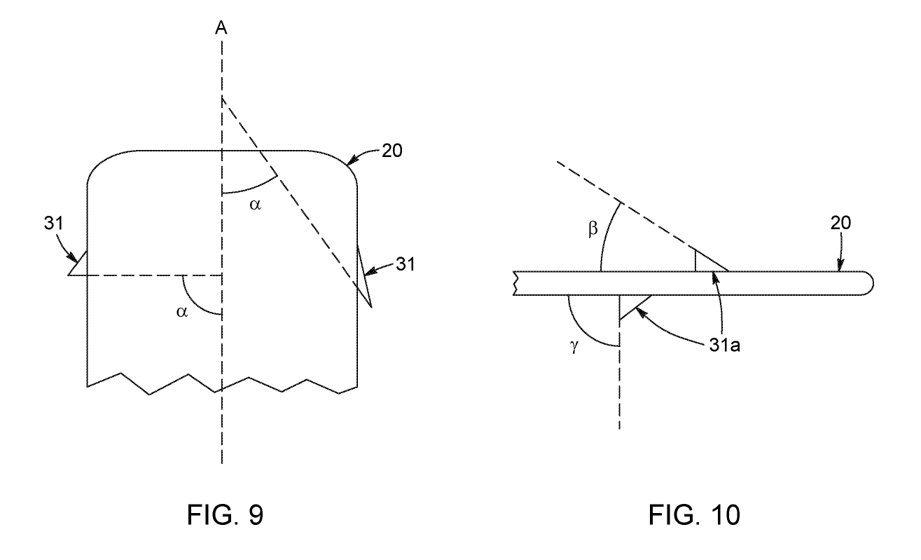

In some implementations, as illustrated in FIG. 9, the angle α formed between the edge spikes 31 and the longitudinal axis A of the applicator is between about 40° and about 50°, or about 45°. It is understood that the angle α may be chosen such that the edge spikes 31 hinder and/or prevent the removal of the absorbent material. The angle α may therefore be lower than 90°.

Referring to FIG. 1, the tab 20 can also include can include at least one surface spike 31a. In some embodiments, each one of the opposing surfaces includes at least one surface spike 31a. In some implementations, the surface spikes are arranged on the tab so that the spikes can engage the sponge and prevent detachment while also not being engaged by the user when the tab 20 is held and twisted or deflected. The surface spikes 31a can have a similar function as the edge spikes 31, which is to hinder and/or prevent the removal of the absorbent material.

It is understood that each spike 31a, 31 can have various shapes which include without being limited to a cone, a prism, a triangular prism and a pyramid. The spikes can for example have a triangular prism shape. One of the surfaces of the triangular prism contacts the surface of the tab, while the other two surfaces are configured to contact the sponge.

In some implementations, as shown in FIG. 10, the angle β between a second surface of the triangular prism and one of the opposite surfaces is about 45°. Similarly, the angle γ between a third surface of the triangular prism and one of the opposite surfaces is about 90°. The angle β can be between 0° and 90° and/or the angle γ can be between 0° and 90°.

In terms of manufacturing the applicator, various methods may be used. In some implementations, the applicator 10 is made by a blow-fill-seal (BFS) method wherein the entire applicator, except the sponge, is formed as an integral one-piece structure and the medical fluid is provided into the chamber during production. As such applicators are mass produced in very large quantities, BFS methods can be preferable. Alternatively, the resilient structure can be a distinct element that is attached to the ampoule body and tab of a pre-made applicator, as generally illustrated in FIG. 4. In this way, known applicators can be “retrofit” by providing appropriate resilient structures and then affixing a sponge to the end. For example, existing applicators that do not have a resilient structure can be modified by attaching a resilient structure thereto. In some implementations, when the resilient structures have certain configurations, it may be desirable to include additional steps after the BFS production step, for example by trimming or punching out certain areas to create desired gaps if necessary (e.g., FIG. 1 illustrates a gap 32 that could be punched out on each side).

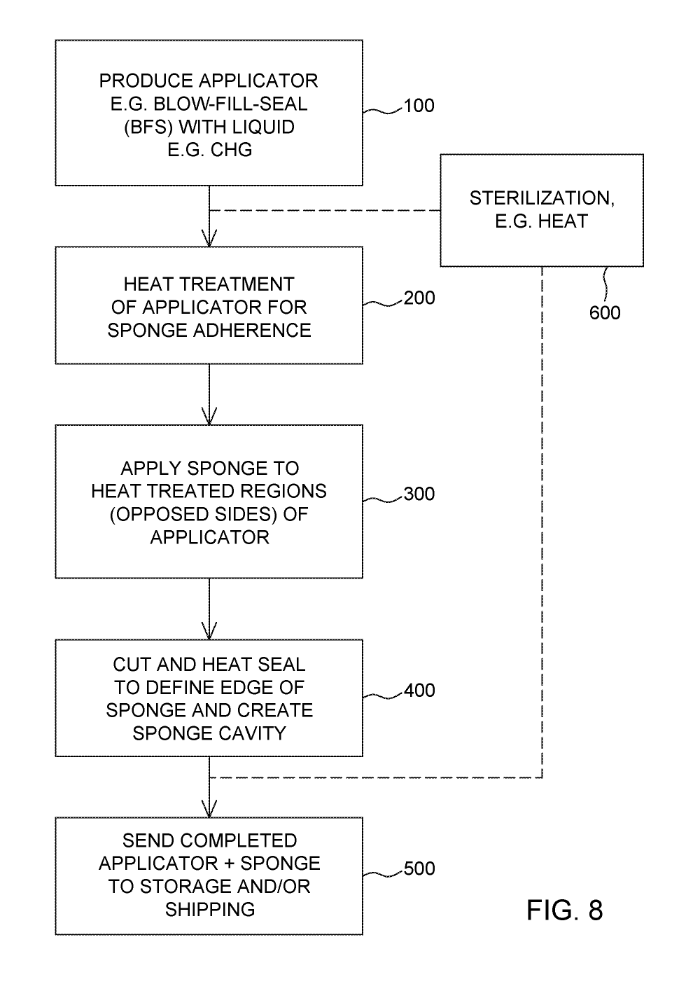

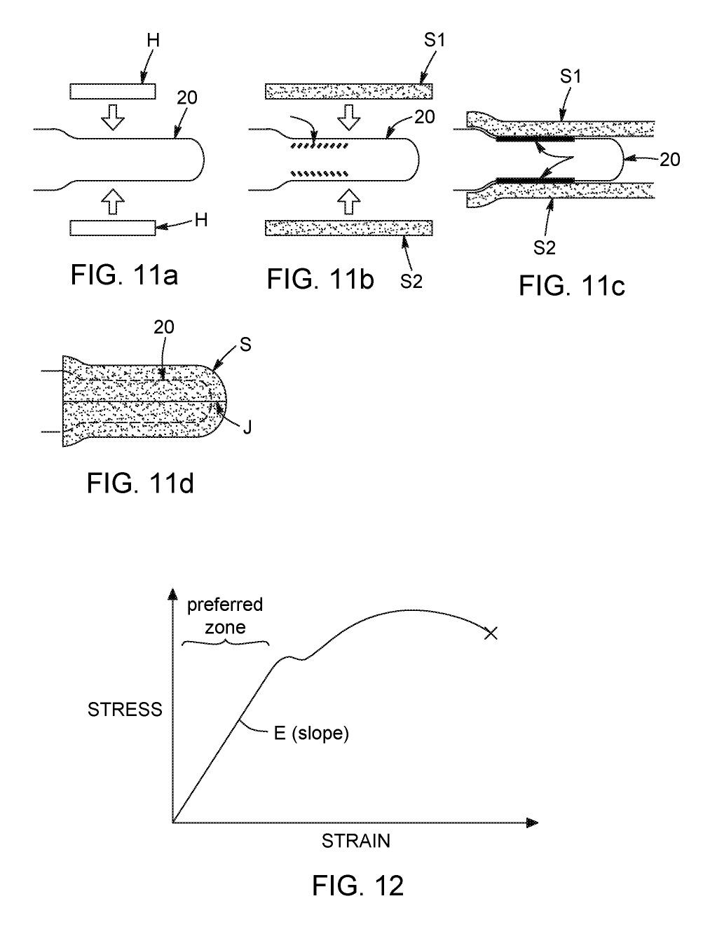

Referring to FIG. 8, the manufacturing the applicator can also include post BFS processing steps in the overall process, for example in order to apply the sponge in an efficient manner. In a first step 100, the applicator is produced by a method such as BFS. In a second step 200, the applicator is heat treated in order to prepare the surface of the applicator for application of the sponge. The heat treatment can be performed on a specific designated area of the applicator, such as the heat treatment regions 34 illustrated in FIG. 3. Preferably, the heat treated regions are limited to at least part of the tab and/or at least part of the distal portion of the ampoule body. The heat treatment should be conducted so that the sponge can be bonded or adhered to the heat treated regions on contact without other adhesives being required. For example, the material can be heated to close to its melting temperature. FIG. 11a shows part of a medical liquid applicator and a heating element H used for the heat treatment. The heating can be done by direct contact or by remote heating methods.

It should be noted that alternative methods can be used to attach the sponge to the applicator, e.g., by using adhesives, glues, etc. at various steps of the production process, and with or without heating.

The third step 300 includes applying the sponge onto the heat treated regions of the applicator to enable adherence. In some implementations, the sponge can be applied in the form of two strips from rolls, each strip being laid on an opposed side of the applicator. This type of sponge strip application can increase throughput and production speed in an automated manufacturing process. Alternatively, the sponges can be prefabricated to have a cavity and can be inserted over the tabs. FIG. 11b shows part of the medical liquid applicator including heat treated regions and sponge strips S₁, S2; and FIG. 11c shows the sponge strips S₁, S2 applied to the heated treated regions so as to bond thereto. The sponge strips S₁, S2 can be fed by roller systems.

In step 400, the two heat-adhered strips of sponge are cut and heat-sealed together to define the edge of the sponge member and to form the cavity of the sponge in which the tab is located. The cutting and heat-sealing can be done by the same processing equipment. FIG. 11d shows the sponge S after cutting and sealing around the sides and far end thus forming a generally U-shaped heat-sealed joint J and defining a sponge cavity. The sponge S is thus pocket shaped and accommodates the tab within the cavity.

In step 500, the applicator with integrated sponge is sent for storage, packaging and/or shipping. Other steps may be performed such as cooling the applicator or various sterilization treatments.

Regarding sterilization of the medical fluid contained in the chamber of the ampoule body, various techniques can be used at different points in the manufacturing process depending on certain factors such as the type of sterilization, the properties of the medical liquid and its constituents, and the material properties of the applicator body and sponge. For example, in the case of heat sterilization, the sterilization treatment step 600 can be performed after making the applicator and before applying the sponge, especially if the sponge material would degrade or undergo undesirable changes upon exposure to the heat treatment. Alternatively, if the sponge can withstand the heat treatment, the step 600 can be performed before or after the sponge application (i.e., before step 200 or after step 400). Other sterilization methods can also be used.

In some implementations, the heat sterilization can be performed according to various techniques, some of which are described in U.S. patent application Ser. Nos. 13/962,317 and 14/150,488, which are incorporated herein by reference. For instance, the heat sterilization can include heating to a temperature above 55° C. and for a sufficient heating time so that sterilization takes place in the liquid as well as any vapour head space within the ampoule body, while avoiding or minimizing degradation of active ingredients and/or any other components of the medical liquid. Various temperatures and times can be used for heat sterilization, such as at least about 62° C. for at least about 12 hours; between about 55° C. and about 70° C. for at least 9 hours; between about 55° C. and about 70° C. for at least 16 hours; between about 55° C. and about 70° C. and the heating time period is at least 24 hours; between about 55° C. and about 70° C. for between 9 hours and 36 hours; between about 60° C. and about 67° C. for at least 12 hours; or at higher temperatures, such as between 85° C. and 135° C. but for shorter time periods.

It should be noted that various sterilization methods can be used notably depending on the type of medical liquid and active ingredients. For instance, gamma sterilization, heat sterilization, and/or ethylene oxide (ETO) sterilization can be used in certain instances. In addition, in some implementations, the medical solution can include iodine-based or chlorhexidine-based (e.g. chlorhexidine gluconate) ingredients. The medical liquid may include one or more solvents. Various solvents may be used. Co-solvents may also be used, as well as various additives. The medical liquid may include one or more excipients. In some scenarios, the medical liquid includes purified water. The medical liquid may include a main active component and an additional active component. In some scenarios, the main active component is chlorhexidine and the additional active component may be an alcohol, which may be a secondary alcohol, such as isopropyl alcohol. The medical liquid may include the main active component (e.g., chlorhexidine) and other components in various proportions. For example, the chlorhexidine may be present in about 0.5% w/v to about 5 t % w/v, optionally about 1% w/v to about 3.5% w/v, or about 1.5 t % w/v to about 2.5% w/v. The additional active component, such as isopropyl alcohol, may be present in about 55% v/v to about 80% v/v, or about 65% v/v to about 75% v/v, for example. Water may be present as the remainder, and/or in about 15% v/v to about 45% v/v or 20% v/v to about 30t % v/v. The medical liquid may contain other additives in various concentrations, for example a dye for tinting the medical liquid and present in an amount sufficient to provide the desired color.

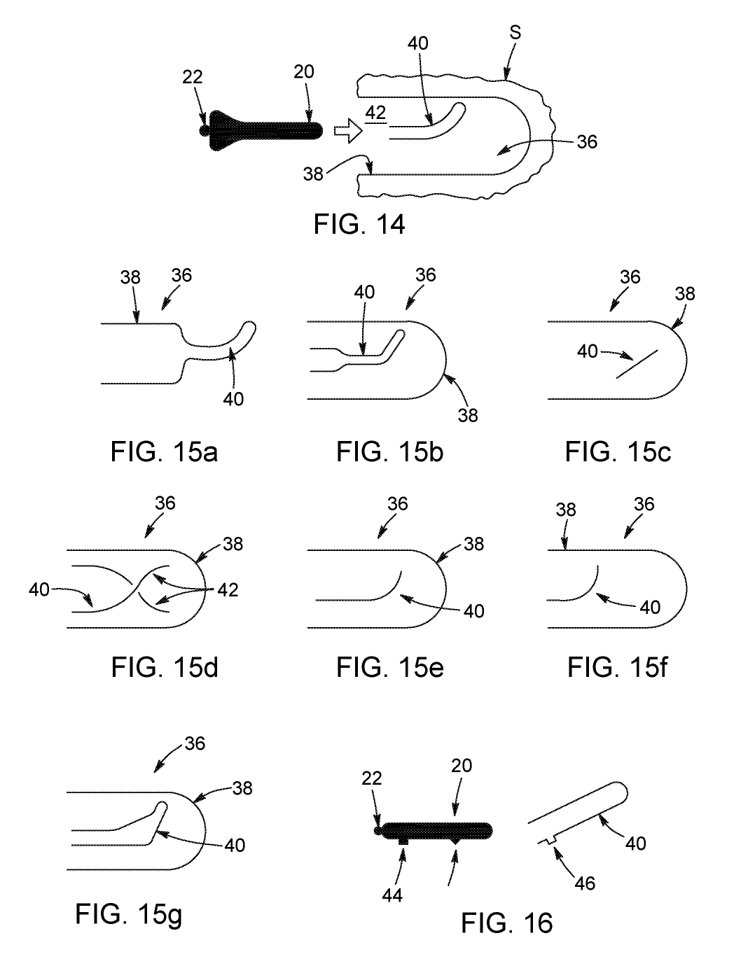

Referring now to FIG. 14, the medical liquid applicator can also include a guide 36 about which the sponge S is mountable and which includes a guide body 38 and a guide mechanism 40. The guide 36 is configured to cause the tab 20 to displace, for example by rotation or deflection, in order to cause breakage of the frangible region 22. In some implementations, the guide 36 defines a cavity 42 into which the tab 20 can be inserted axially, wherein the tab 20 engages the guide mechanism 40 causing the tab 20 to deviate from its straight orientation as the tab 20 is inserted. Eventually, the tab 20 is sufficiently deviated to cause the frangible region to break and release medical liquid into the guide body 38 and into the sponge. The guide body 38 may thus be configured with openings or have an open structure so that the liquid can flow into the sponge S. The guide body 38 may also have an outer surface to which the sponge S is adhered or otherwise attached by various methods, including gluing, heat bonding, and so on. The guide body 38 may have spikes similar to those described herein for the tab 20, in order to anchor the sponge S to the guide body 38.

In some implementations, the tab 20 and the guide mechanism 40 are configured such that when the tab 20 is inserted, the tab 20 contacts part of the guide mechanism 40 which causes deflection of the tab relative to the longitudinal axis. Thus, the applicator can be displaced axially so the tab 20 moves into the guide 36, and the tab 20 is forced sideways by the guide mechanism which has a curvature or a deviated structure. The guide mechanism 40 is configured to cause sufficient deviation of the tab 20 to cause breakage of the frangible region 22.

In some implementations, the guide mechanism 40 can be in the form of a groove, recess or projection provided in the inner surface of the guide body 38, as schematically illustrated in FIGS. 14, and 15b to 15g. The side edges of the tab can cooperate with the groove or recess to slide along the desire deviated path to induce breakage of the frangible region. When the guide mechanism is a projection, which may take the form of a small wall projecting from an inner surface of the guide body 38, the tab can contact the projection and be deviated. Alternatively, the guide body 38 can itself have a shape and configuration to have a portion that defines the guide mechanism, as schematically illustrated in FIG. 15a.

In some implementations, the guide mechanism 40 can have a curvature, as illustrated in FIGS. 14, 15a, 15d, 15e, and 15f, or can include one or more straight sections as illustrated in FIGS. 15b, 15c, 15g and 16. The guide mechanism 40 have be located close to a forward end of the guide body, as in FIG. 15e, or at a rearward end of the guide body as in FIG. 15f. In addition, the guide mechanism 40 can be configured to cause deflection of the tab, i.e. bending with respect to the axis of the applicator, or to cause rotation of the tab within the guide body for example using helically shaped guiding elements 42 as schematically shown in FIG. 15d.

Referring now to FIG. 16, in some implementations the guide mechanism 40 and the tab 20 can include a locking mechanism that enables the tab and guide mechanism to be locked once the tab has reached a broken position, i.e., in which the frangible region has broken. For example, the locking mechanism can include at least one protrusion 44 and at least one corresponding recess 46 on the tab and guide mechanism positioned to cooperate and lock the two parts together to prevent the tab from being withdrawn. The protrusion 44 can be on the tab or the guide mechanism, and the recess 46 can be on the other component. The positioning of the protrusion 44 and the recess 46 is such that they engage once the frangible region is broken. There may also be a pre-locking mechanism, which may include a clicking-element 48 that engages with another element to make a clicking sound to indicate to the user that the engagement of the tab and the guide mechanism has initiated but breakage has not yet occurred. For instance, the clicking-element 48 may be a projection mounted to the tab forward of the protrusion 44 and provided with a shape and size so that the protection 48 partially engages with the recess 46 (or another element provided on the guide mechanism) and makes a sound or another indication of engagement such as a slight resistance to insertion. Of course, various other locking mechanism configurations can be provided depending, for example, on the configuration and functionality of the guide mechanism and the tab.

Furthermore, when the applicator includes the guide 36, the resilient structures can be sized, positioned and configured accordingly. For instance, the resilient structures can extend along a certain length of the tab or may have a certain thickness or shape to facilitate cooperation of the tab and the guide mechanism. In some implementations, the applicator may include the guide 36 while not having the resilient structures integrated with the tab and ampoule body, as described herein. Rather, the guide 36 may have the configuration and structural properties such that once the tab is inserted and the frangible region is broken, the guide 36 can provide axial rigidity to resist axial deflection in response to a deflection pressure exerted during application of the medical liquid. In such scenarios, the guide 36 is coupled or locked with respect to the rest of the applicator once the broken position is attained. Thus, the locking mechanism may be configured in various ways so that the sponge has the desired structural resistance to axial deflection during liquid application. The locking mechanism may be integrated with the guide mechanism and the tab for locking between those components, as described above and illustrated in FIG. 16, but it should be understood that the locking mechanism may also be attached to or associated with various other components of the applicator (e.g., the tab, the ampoule body, the guide body, the guide mechanism) to enable locking of the guide with respect to the ampoule body gripped by a user during operation.

In some implementations, the guide body 38 has a proximal end that defines the opening through which the tab is inserted. Prior to displacing the tab toward the breaking position, the guide body may encompass the entire tab and/or part of the ampoule body. In addition, after the tab has been displaced to the breaking position, the guide body should encompass the entire tab and part of the ampoule body. The proximal end may be sized such that the opening can also receive part of the ampoule body, if desired, and in some scenarios for a relatively close fit of the ampoule body within the end of the cavity. The cooperation and the fit of the ampoule body and the guide may be sufficient to provide the axial rigidity to resist the axial displacement during liquid application, thus providing the resilient structure. A locking mechanism may be provided to lock the guide body with respect to the ampoule body in the broken position.

In some implementations, the applicator can be packaged and/or sold with two separate parts: (1) the ampoule body and tab part and (2) the guide and sponge part. In use, the guide and sponge part is engaged with the ampoule body and tab part as explained above. Alternatively, the two parts can be packaged and/or sold as a pre-assembled construction, for example where the guide and sponge part is pre-fit over the tip of the tab and can optionally be held in place by friction or another mechanism. In use, the tab is further displaced within the guide as explained above.

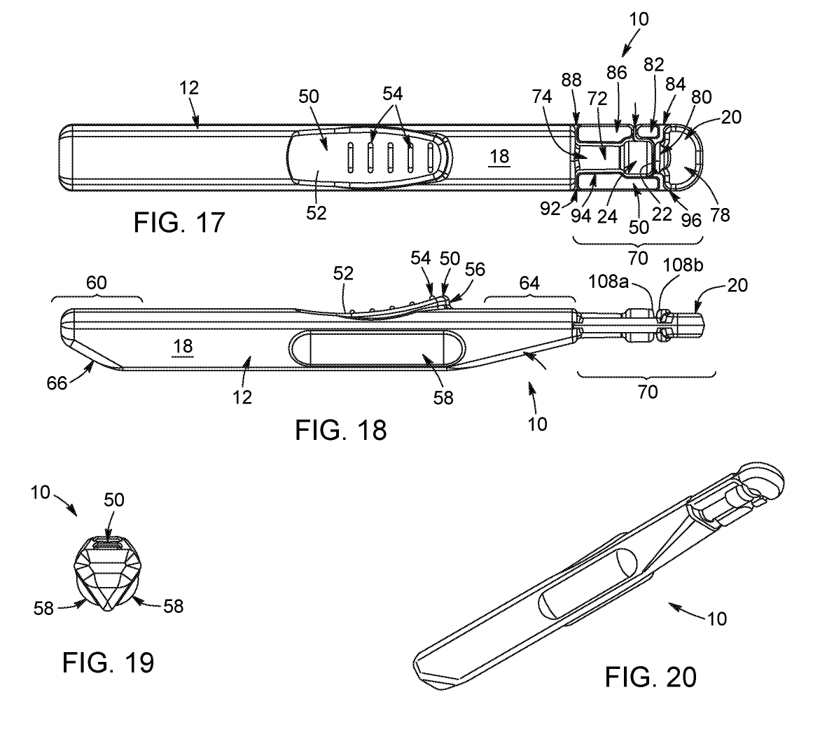

Referring to FIG. 17, in some implementations the applicator 10 can include a thumb rest 50 disposed on an upper side of the ampoule body 12. The thumb rest 50 may have a generally oval or stadium shape, and may include an upward-facing surface 52 having a concave curvature. In some implementations, the curvature has a generally constant radius of curvature along its length, and may be for example about 2 cm to 4 cm, 2.5 cm to 3.5 cm, or 3 cm long. The curvature of the thumb rest 50 may be configured to have a 40° to 10° arc or a 30° to 20° arc, for example. The thumb rest 50 may also have ridges 54 extending laterally across part of the upward-facing surface to provide friction and grip for the user. The ridges 54 may be provided only at the forward portion of the thumb rest 50 leaving the rearward portion of the thumb rest 50 relatively smooth.

Referring to FIG. 18, the thumb rest 50 may have a forward end lip 56 that is raised, for example by about 2 mm to 3 mm, above the adjacent main wall of the ampoule body 12. The remainder of the thumb rest 50 may be below this forward end lip 56.

Referring still to FIG. 18, in some implementations the wall of the ampoule body 12 can define at least one bubble portion 58, which can be provided on an opposed or adjacent side of the ampoule body 12 as the thumb rest, i.e., on a bottom side. When the ampoule body 12 has a generally triangular cross-section and has three main side walls in its central region, there may be two bubble portions 58 on the two bottom sides, as shown in FIG. 19. The bubble portions 58 can each be generally elongated and can have a stadium shape. In some scenarios, the bubble portions 58 can each have a length that is generally the same as the thumb rest 50. The bubble portions 58 can each protrude from the main flat part of the wall of the ampoule body 12 to define an internal bubble volume that is part of the ampoule chamber for holding the medical liquid. When a user exerts a compressive force by pinching the ampoule body between thumb and opposing finger(s), the bubble portions 58 are compressed and facilitate dispensing of the liquid. In general, larger bubble portions will facilitate greater volumes of liquid to be dispensed per squeeze.

Referring to FIG. 18, the ampoule body 12 can have a structure that provides various functionalities, including axial rigidity and spacing relative to an application surface. In some implementations, the ampoule body 12 has a rear region 60, a central region 62, and a forward region 64. The central region 62 can include the thumb rest 50 and the bubble portions 58. The rear region 60 can be tapered and include an inclined bottom wall 66. The inclined bottom wall 66 may have a generally triangular shape with the wider side toward the rear, and may be generally flat. The forward region 64 can also include a second inclined bottom wall 68 that is generally similar to the first inclined bottom wall 66, although different structures, configurations and angles are possible. In some scenarios, the second inclined bottom wall 68 is configured and oriented to provide greater space between the forward region 64 of the ampoule body 12 and the application surface during application of the medical liquid.

Referring to FIG. 19, the ampoule body 12 may include a construction where it has a triangular cross-section along a substantial part of its length. In some instances, the central region 62 of the ampoule body 12 may have a triangular cross-section, while the rear region 60 and the forward region 64 have trapezoid shapes owing to their tapering configurations. The triangular cross-section structure can facilitate axial strength along the length of the ampoule body 12 to reduce excessive bending during liquid application. The cross-sectional shape may be generally triangular (e.g., isosceles or equilateral) with the walls having rounded corners.

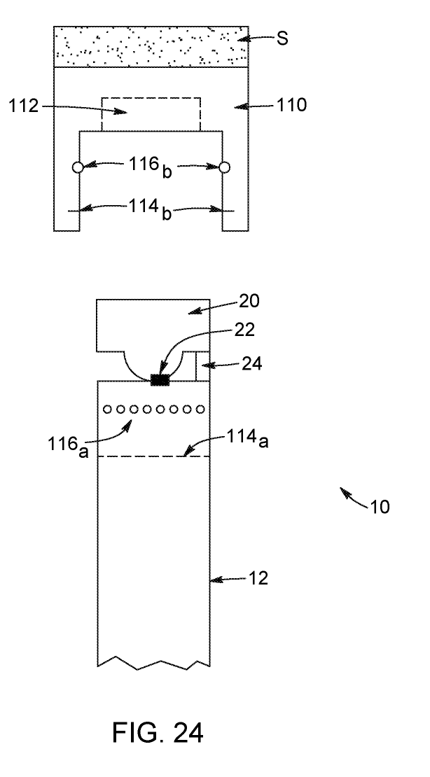

In some implementations, the ampoule body 12 may include a section that is generally cylindrical to facilitate cooperation with other optional components. For example, the ampoule body 12 may include a cylindrical section at the forward region 64 so that a head component can be arranged over the tab and a connecting mechanism disposed on the head component and the cylindrical section of the ampoule body can cooperate to enable axial and/or radial locking between the ampoule body and the head component. For example, features related to head component and its connection and interaction with the ampoule body can be used or adapted from international application No. PCT/CA2015/050436, which is incorporated herein by reference. More regarding this scenario will be discussed below with reference to FIG. 24.

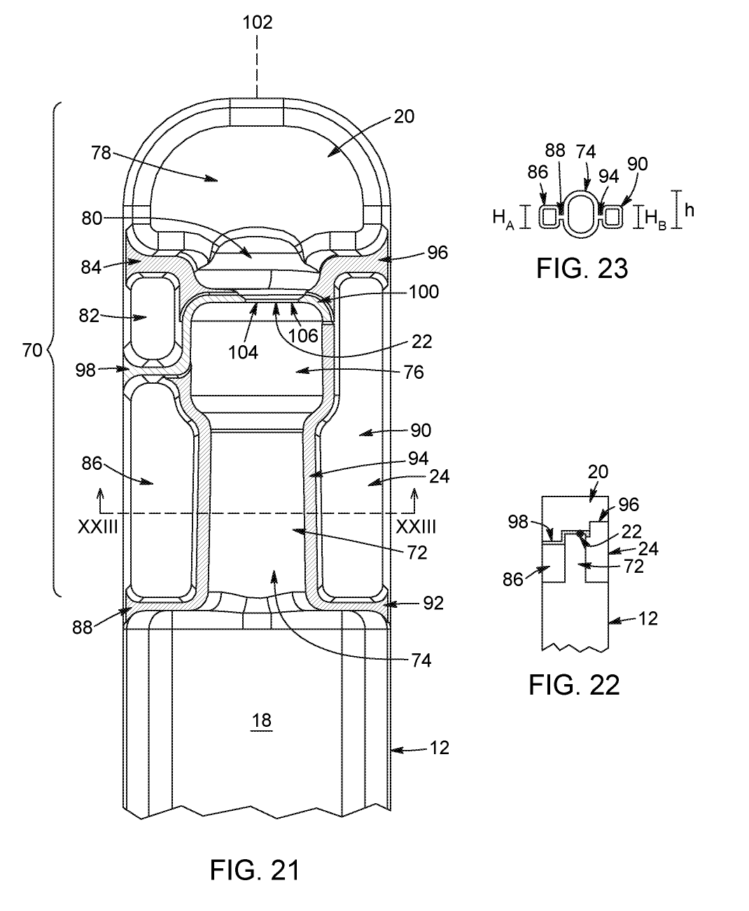

Referring to FIGS. 17 and 21, in some implementations the applicator 10 can have certain structural and functional features in the region generally referred to herein as a “head region” 70. It is noted that the head region 70 can include parts of the ampoule body 12, the tab 20, the frangible region 22 or nodule, and the resilient structure 24. The head region 70 can be formed pursuant to a molding technique use to make the applicator. In general, the head region 70 can include various structures, such as cellular units which each have a wall defining an inner chamber and thus have a shell or enclosure type structure and which are interconnected to other components of the head region via rigid webs (e.g., thick webs) and frangible webs (e.g., thin webs) to enable desired functionality.

Referring to FIGS. 17 and 21, the head region 70 can include an ampoule neck 72 that extends forwardly from the main part of the ampoule body 12 and defines a neck chamber that is in fluid communication with the ampoule body chamber 18. The ampoule neck 72 can include a rearward neck portion 74 and a foreword neck portion 76, which can both be generally cylindrical. The rearward neck portion 74 can be thinner than the forward neck portion 76, e.g., the former can have a length greater than its diameter (e.g., about 1.75 to 2.25 times greater) while the latter may have a length and diameter that a generally equal or similar. The two neck portions 74, 76 can also have rounded edges.

As shown in FIGS. 17 and 21, the frangible region 22, which may include or take the form of a frangible nodule, is disposed between the tab 20 and the forward neck portion 76 of the ampoule body 12. The frangible region 22 may interconnect an upper wall of the forward neck portion 76 and a lower wall of the tab 20. The tab 20 may also have a configuration to include a distal portion 78 and a proximal portion 80, wherein it is the lower wall of the proximal portion 80 that is interconnected to the forward neck portion 76. The distal and proximal portions 78, 80 of the tab 20 can form a single cellular unit. In some implementations, the distal and proximal portions 78, 80 are both symmetrical about a central longitudinal axis. In some implementations, the distal and proximal portions 78, 80 have different heights, wherein the proximal portion 80 has a greater height than the distal portion 78. In some scenarios, the distal portion 78 has a generally disk shape viewed form the side, and the proximal portion 80 has a bulbous shape. In some implementations, the proximal portion 80 has a generally similar height as the forward neck portion 76. The proximal portion 80 and the forward neck portion 76 can also have opposed surfaces that face each other and are sized, positioned and configured to abut on each other when the tab is deflected, for instance in response to a force when applying the medical fluid.

Referring still to FIGS. 17 and 21, the tab 20 may include a side member 82 that extends rearwardly (i.e., toward the rear of the applicator) and may extend adjacent to part of the ampoule neck 72. The side member 82 may include cellular unit that is connected to the distal and proximal portions 78, 80 of the tab 20 via a rigid tab web 84. The rigid tab wed 84 enables the side member 82, the distal portion 78 and the proximal portion 80 of the tab to form a single structural unit that can rotate together.

FIGS. 17 and 21 also illustrate a lateral member 86 that is arranged adjacent to the ampoule neck 72, and is connected to the ampoule body 12 via another rigid web 88. This lateral member 86 can provide additional rigidity to the ampoule neck 72 to resist bending for example. The lateral member 86 can also include cellular unit.

FIGS. 17 and 21 further illustrate that the resilient structure 24 that connects the ampoule body 12 to the tab 20 can include several components including a resilient cellular unit 90, a proximal web 92, a side web 94, and an end web 96. The proximal and side webs 92, 94 connect the resilient cellular unit 90 to the ampoule body, e.g., to the main ampoule body and the ampoule neck respectively. The end web 96 connects the resilient cellular unit 90 to the tab 20, e.g., to a lower part of the tab 20 that is adjacent to the frangible region 22. The webs 92, 94, 96 can each have sufficient rigidity to provide the functions of connection with the other components and resistance to bending. For example, the webs 92, 94 can have structural properties (e.g., thickness, density, material, size, configuration, etc.) to solidly connect the resilient cellular unit 90 to the ampoule neck and main ampoule body. In addition, the end web 96 can have structural properties that facilitate rotation of the tab 20 to break the frangible region 22 while maintaining a solid connection between the tab and the resilient cellular unit 90 (e.g., to prevent breaking therebetween, to provide resistance to bending during fluid application, etc.).

In some implementations, the cellular units 86 and 90 are arranged beside and along the ampoule neck 77 and they are sized and shaped such that their inner surfaces follow the contour of the ampoule neck 72 and the main ampoule body. Thus, webs 88, 92, 94 have general constant thickness along their length.

Referring still to FIG. 17, the head region 70 may also include a frangible web that is disposed in between the tab 20 and adjacent components. In some implementations, the frangible web includes a first frangible web 98 that is located in between the tab 20 and the ampoule body 12, e.g., in between the side member 82 and the lateral member 86, in between the side member 82 and the ampoule neck 72, and in between the proximal portion 80 and the ampoule neck 72. The first frangible web 98 may extend continuously from a lateral edge to the frangible region 22, thereby defining a frangible path that is torn as the tab is rotated relative to the ampoule body. The frangible web may also include a second frangible web 100 that is located in between the tab 20 and the ampoule body 12 and optionally the resilient structure 24, e.g., in between the end web 96, the side web 94 and the ampoule neck 72. The second frangible web 100 may provide some rotational flexibility during rotation of the tab and may also shear after breaking the frangible region 22. The frangible webs 98, 100 may be composed of relatively thin plastic material. In some alternative implementations, the regions defined by some or all of the frangible web 98 and/or 100 may include perforations or only periodic connection points to facilitate tearing or may be void spaces where no material is present.

Referring still to FIGS. 17 and 21, the frangible web 98 that extends from the lateral edge to the frangible region 22 can have a generally constant thickness along its length and can define different paths that enable the tab to be rotated to break the frangible region. In some implementations, the frangible web 98 includes a first web section that extends inwardly from the edge, a second web section that extends forwardly toward the distal tip of the applicator, and a third web section that extends inwardly to the frangible region 22. The frangible web can have a zigzag shape. The second web section can be relatively short so as not to provide too much resistance to rotation. In some scenarios, the first and third web sections may be provided, along with any other laterally extending web sections, and any longitudinally extending web section are excluded and replaced with void space. In some scenarios, the frangible web 98 can also include web sections that are angled relative to longitudinal or lateral axes. The frangible web 98 and the frangible region 22 are preferably configured, sized and arranged to enable sequential breakage of the web 98 followed by the region 22, and then optionally followed by the second frangible web 100 on the opposed side.

As described above and referring to FIG. 23, in some implementations the head region can include cellular units which form certain components of the applicator. The cellular units can facilitate certain structural properties, such as resistance to bending or torsion forces while using an efficient amount of material. Each cellular unit is preferably a single-cell structure having a thin wall enclosure that facilitates distribution of stress. The cellular units, such as cellular units 86 and 90 illustrated in FIG. 23, each have dimensions that include a height component H_{A}, H_{B} as well as width and length components, which facilitate resistance to axial bending during application of the medical fluid. Other components of the applicator, such as the ampoule neck, may also have dimensions that include a notable height component (h) to resist such bending. The cellular units can also resist other forces, such as torsion forces, that may be experienced during use when the tab and ampoule body are rotated relative to each other. Thus, whereas flat solid parts may buckle under forces or require significant quantities of material to achieve desired structural properties, the cellular units are able to provide enhanced structural properties with lower usage of material due to the thin wall construction.

In some implementations, the cellular units also define surfaces for connection with respect to the sponge, which may be done using heat, adhesives, or other means.

Referring to FIG. 17, the frangible region 22 may be located offset from a central longitudinal axis 102 of the applicator and is located closer to the pivot point or hinge that exists during rotation of the tab 20. In some implementations, the frangible region 22 is fully offset and does not cross the central longitudinal axis 102, while in other implementations the frangible region 22 has a first portion 104 that crosses the axis 102 and a second portion 106 that is on the opposed side of the axis 102. By providing the frangible region 22 offset from the central axis and closer to the pivot point for rotation, greater leverage can be achieved for rotating the tab relative to the ampoule body.

While the Figs illustrate a single frangible nodule which, when broken, form an opening for fluid communication out of the ampoule body (e.g., via the ampoule neck), it should be noted that there may be multiple frangible nodules located in between the tab and the ampoule body (e.g., preferably at the tip of the neck). For example, there may be two or more distinct frangible nodules which, when broken, for one common opening or multiple distinct openings for fluid flow. The frangible nodules can be identical in shape and size, or they can have different shapes or sizes (e.g., nodules that are located further away from the pivot point can be smaller to facilitate breakage in response to rotation). When deflection is used to break the frangible region, the location of one or more frangible nodules can be provided accordingly.

In some alternative implementations, the applicator may not include a resilient structure extending between the tab and the ampoule body. Such applicators can include one or more features described herein, which may include a thumb rest, structural features of the ampoule body and/or its neck, cellular unit structure of various components like the tab or adjacent members in the head region, location (e.g., offset) of the frangible region, location or configuration of various rigid and/or frangible webs, etc. In some implementations, the ampoule neck provides structural rigidity to resist bending while the tab can have some degree of bending in response to downward application forces. Referring to FIG. 18, in some implementations the ampoule neck and the tab have opposed abutment surfaces 108a, 108b which may abut against each other if the tab 20 bends during fluid application, thereby inhibiting further deflection of the tab.

Referring to FIG. 24, the applicator 10 may include a head component 110 to which the sponge (S) is attached. The head component 110 may include a slot 112 sized and configured to receive at least a part of the tab 20. The head component 110 and the applicator 10 may also include locking mechanisms, which may include axial and/or radial locking mechanisms. The axial locking mechanism may include two cooperating parts 114a, 114b which connect to axial lock the head component to the ampoule body 12. The radial locking mechanism may include two cooperating parts 116a, 116b which connect after rotation of the head component relative to the ampoule body 12 and after breakage of the frangible region 22. Thus, in operation, the ampoule body is axially locked into place so that the tab 20 is located in the slot 112 of the head component 110, and then the head component 110 can be rotated in order to cause the tab 20 to rotate relative to the ampoule body 12 until the frangible region 22 is broken and fluid is allowed to flow. At breakage, the radial locking mechanism radially locks the head component 110 with respect to the ampoule body 12, thereby preventing additional rotation of the head component 110. The head component 110 also includes a flow channel allowing fluid communication between the breakage opening and the sponge (S). Various other optional features of the head component, ampoule body, tab and other parts of the applicator can be adapted from PCT/CA2015/050436.

In terms of manufacturing the applicator, various methods can be used. In some implementations, a molding technique is used. For example, a parison can be molded into one or more applicators. In some scenarios, blow molding, injection molding, blow-fill-seal (BFS) molding or a combination thereof can be used to manufacture the applicator. In some preferred scenarios, each applicator is a single integral one-piece structure of molded plastic. The materials that can be used include various polymeric materials, such as high density polyethylene (HDPE), polyethylene terephthalate (PET), polypropylene (PP), and so on. The material composition of the applicator can be provided in accordance with the desired application, e.g., in order to ensure regulatory requirements, inhibit chemical interaction between the material and the fluid, ensure a fluid seal that is appropriate for the given fluid, and so on. In some alternative implementations, the applicator can include several components that are independently manufactured (e.g., by molding) and then connected together for operations (e.g., by threaded connections, adhesives or other connection mechanisms that can enable a fluid seal if necessary).

It is also noted that the FIGS. 17 to 21 can be considered as disclosing the actual relative dimensions, orientations, and positions of the components of an example applicator.

Embodiments of the applicator or general device features can be used in various end-uses. In some implementations, the applicator can be used for applying medical fluids to a surface, such as human epidermis in various contexts including surgery. In some implementations, the device can be used for dispensing fluids that include a pharmacological or medicinal component, such that the fluids are expelled by dripping, jetting or other fluid dispensing mechanism that do not necessitate a sponge. In some implementations, other uni-dose liquids can be held and dispensed using the applicator device disclosed herein. In some implementations, the applicator device can be used to dispense a cleaning fluid for various applications, such as industrial, domestic or medical cleaning applications. In some implementations, the applicator can be used for industrial or laboratory applications for dispensing fluids. In some implementations, the applicator can be used for dispensing consumables, such as liquids or suspensions that can be ingested, such as energy gels, drinks, and so on. In some implementations, the applicator can be used for artistic applications where the fluid includes a chromatic material such as paint. The fluid that can be accommodated and dispensed by the applicator can include liquids, solutions (liquids with dissolved compounds), suspensions (liquids with suspended solid particulates), gels, multi-phase fluids or dispersions, and so on. While the present specification may refer to “medical liquids” when describing the applicator, it should be noted that various other fluids in other applications can be implemented with the applicator which may include one or more features as described herein.

The applicator may have various configurations, shapes and sizes, which also may depend on the desired application. For example, the applicator can be sized to accommodate various volumes of liquid (e.g., 1 ml to 50 ml).

It should be understood that various other shapes, sizes, configurations of the applicator components not described or illustrated can also be used.

1. A medical liquid applicator, comprising:
an ampoule body comprising a proximal portion and a distal portion, and having a wall defining a chamber for holding a medical liquid;
a tab attached to and extending away from the distal portion of the ampoule body, the tab being twistable about or deflectable with respect to a longitudinal axis of the ampoule body;
a frangible region in between the tab and the distal portion of the ampoule body, the frangible region being breakable in response to twisting or deflection of the tab to form an opening in fluid communication with the chamber to allow liquid communication out of the chamber into an absorbent material connectable to the tab; and
a resilient structure extending between the tab and the ampoule body and configured to allow the twisting or deflection of the tab to enable breakage of the frangible region while remaining intact and providing axial rigidity to resist axial deflection of the tab in response to a deflection pressure exerted on the tab during application of the medical liquid.

2-107. (canceled)

108. The medical liquid applicator of claim 1, wherein the resilient structure comprises at least two resilient structures provided on opposed lateral sides of the tab and connecting to opposed lateral sides of the distal portion of the ampoule.

109. The medical liquid applicator of claim 108, wherein the resilient structures each comprise an arm having a first portion extending from the tab along and in spaced-apart relation to the distal portion of the ampoule and a second portion extending toward and attaching to the distal portion of the ampoule.

110. The medical liquid applicator of claim 108, wherein the resilient structures each comprise a thick zone integrally formed with the tab and the distal portion of the ampoule body.

111. The medical liquid applicator of claim 1, further comprising the absorbent material that is affixed to with respect to the tab, wherein the absorbent material comprises a sponge having opposed sides and a cavity housing the tab, wherein the sponge is heat-bonded to part of the tab and/or part of the distal portion of the ampoule body and is further retained on the tab by engaging with retention elements including spikes provided on the sponge.

112. A medical liquid applicator, comprising:
an ampoule body comprising a proximal portion and a distal portion, and having a wall defining a chamber for holding a medical liquid;
a tab attached to and extending away from the distal portion of the ampoule body, the tab being twistable about or deflectable with respect to a longitudinal axis of the ampoule body;
a frangible region in between the tab and the distal portion of the ampoule body, the frangible region being breakable in response to twisting or deflection of the tab to form an opening in fluid communication with the chamber to allow liquid communication out of the chamber; and
at least one bubble portion provided on a bottom side of the ampoule body, each bubble portion comprising a bubble wall that is continuous with the wall of the ampoule body and projects to form an elongated bubble compartment that is configured to facilitate fluid dispending in response to compression of the ampoule body.

113. The applicator of claim 112, wherein the ampoule body comprises a main section having a generally triangular cross-section so as to provide an upper wall and two bottom inclined walls, and the at least one bubble portion comprisse two bubble portions arranged on the two bottom inclined walls.

114. The applicator of claim 112, wherein each bubble portion is provided in opposed relation to a thumb rest disposed on an upper side of the ampoule body and comprises an upward-facing surface having a concave curvature.

115. The applicator of claim 112, wherein each bubble portion has a volume between 50 mm³ and 300 mm³.

116. A medical liquid applicator, comprising:
a body region comprising a main container portion having a wall defining a chamber for holding a medical liquid; and
a head region coupled to the body region and comprising:
a neck portion extending forwardly from a distal end of the main container portion and defining a neck chamber in fluid communication with the chamber of the main container portion;
a cellular tab unit coupled to and extending forwardly away from a distal end of the neck portion;
a frangible nodule located in between the cellular tab unit and the neck portion, and being breakable in response to rotation of the cellular tab unit relative to the neck portion to form an opening in the neck portion to allow the medical liquid to flow there-through;
a first lateral cellular unit and a second lateral cellular unit disposed on opposing sides of the neck portion and extending lengthwise along at least a part of the neck portion;
a first rigid web disposed between and joining the first lateral cellular unit to the neck portion and the main container portion;
a second rigid web disposed between and joining the second lateral cellular unit to the neck portion and the main container portion; and
a frangible web extending from a lateral edge of the head region above the first lateral cellular unit, inwardly to the frangible nodule, the frangible web being tearable in response to rotation of the cellular tab unit relative to the neck portion such that upon rotation:
the frangible web tears from the lateral edge to the frangible nodule; and
then the frangible nodule breaks to form the opening in the neck portion for fluid flow out therefrom.

117. The applicator of claim 116, wherein the first lateral cellular unit comprises a distal extremity that is located rearward of the neck portion.

118. The applicator of claim 117, wherein the head region further comprises:
a third lateral cellular unit located on a distal side of the frangible web; and
a third rigid web coupling the third lateral cellular unit to the cellular tab unit.

119. The applicator of claim 118, wherein the second lateral cellular unit extends longitudinally from the main container portion to the cellular tab unit and is connected to the cellular tab unit by a forth rigid web.

120. The applicator of claim 116, wherein the head region further comprises a secondary frangible web extending from the frangible nodule in between a distal corner of the neck portion and a distal inner side of the second lateral cellular unit.

121. The applicator of claim 116, wherein the frangible web defines a zigzag path from the lateral edge to the frangible nodule.

122. The applicator of claim 116, wherein the first and second lateral cellular units have generally straight outer walls and inner walls that are shaped to follow an outer contour of the neck portion.

123. The applicator of claim 116, wherein the first and second lateral cellular units each have proximal portions and distal portions, the proximal portions being wider than the distal portions.

124. The applicator of claim 116, wherein the cellular tab unit comprises a distal part and a proximal part, the distal part being wider than the proximal part.

125. The applicator of claim 124, wherein the distal part has a generally circular segment shape viewed from above with a forward curved surface, and the proximal part has a generally circular segment shape viewed from above with a rearward curved surface.

126. The applicator of claim 116, wherein:
each of the cellular tab unit, the first lateral cellular unit, and the second lateral cellular unit is substantially symmetrical about a longitudinal cross-sectional plane extending through the applicator;
the head region and the body region together have generally continuous lateral edges viewed from above; and
each of the cellular tab unit, the first lateral cellular unit, and the second lateral cellular unit has a single-cell structure.
